(19) United States
(12) Patent Application Publication
(10) Pub. No.: US 2022/0096407 A1
(43) Pub. Date: Mar. 31, 2022

(54) COMPOSITION FOR INHIBITING RESPIRATORY VIRUSES AND RESPIRATORY VIRUS PREVENTION AND TREATMENT METHOD

(71) Applicant: Zongzhan DU, Jinan (CN)

(72) Inventors: Zongzhan DU, Jinan (CN); Qi GAO, Jinan (CN)

(73) Assignee: Zongzhan DU, Jinan (CN)

(21) Appl. No.: 17/034,787

(22) Filed: Sep. 28, 2020

(51) Int. Cl. A61K 31/19; A61K 47/02; A61K 47/10; A61K 45/06; A61K 39/00; A61K 31/138; A61K 31/5415; A61K 31/4402; A61K 31/352; A61K 31/4535; A61K 31/47; A61K 31/404; A61K 9/00; A61P 31/14; A61P 31/12; A61P 31/10; A61P 31/04; A61P 11/00; A61P 31/06

(52) U.S. Cl. CPC A61K 31/19; A61P 31/06; A61K 47/10; A61K 45/06; A61K 39/0003; A61K 31/138; A61K 31/5415; A61K 31/4402; A61K 31/352; A61K 31/4535; A61K 31/47; A61K 31/404; A61K 9/007; A61P 31/14; A61P 31/12; A61P 31/10; A61P 31/04; A61P 11/00; A61K 47/02

(57) ABSTRACT

A composition inhibits respiratory viruses and a method prevents and treats a respiratory. Respiratory viral infections are extremely common clinically, but the current therapeutic drugs for viral infections still cannot meet the treatment needs. An inhalant inhibits respiratory viruses and adopts a method of inhaling high-temperature acetic acid steam for prevention and treatment. Acetic acid in the high-temperature steam can release hydrogen ions to inactivate viruses in the respiratory tract, so as to prevent the onset of the disease and provide a therapeutic effect. In addition, the composition can be used to inhibit fungi and bacteria in vitro and prevent skin diseases and respiratory diseases caused by bacteria and fungi. The prevention and treatment method is easy to implement and has low costs and important clinical application value.

### BACKGROUND

### Technical Field

The present invention belongs to the technical field of biomedicine, and specifically relates to a composition for inhibiting respiratory viruses and a respiratory virus prevention and treatment method.

### Related Art

Disclosure of the related art information is only intended to increase the understanding of the overall background of the present invention, and is not necessarily regarded as an acknowledgement or in any form suggesting that the information constitutes the prior art known to those of ordinary skill in the art.

As one of the common clinical diseases, respiratory viral infectious disease has complex etiology and high viral variability, which is prone to outbreaks in the population. At present, there are as many as 100-200 kinds of viruses that can cause respiratory infections, which can be divided into RNA viruses and DNA viruses. The most common pathogenic viruses include influenza viruses, parainfluenza viruses, respiratory syncytial viruses, adenoviruses, rhinoviruses and coronaviruses. In recent years, there have been several new highly pathogenic viruses which mainly infect the respiratory tract, such as SARS coronavirus (SARS-CoV), H5N1 human avian influenza A virus and 2009 novel H1N1 influenza A virus. Due to the aging society, organ transplantation, the application of immunosuppressants in immunity-related diseases, the increase in the incidence of human acquired immunodeficiency syndromes, the increasing number of patients and other factors, the incidence of new or recurring respiratory viral infections is increasing, and the fatality rate of some viral infections is extremely high. Therefore, this type of diseases has become a public health problem which cannot be ignored.

Novel coronavirus pneumonia (Corona Virus Disease 2019, COVID-19) refers to the pneumonia caused by the 2019 novel coronavirus infection. The structure of the novel coronavirus includes a protein coat and a single-stranded RNA carrying genetic materials. This simple structure reduces the difficulty of replication of the novel coronavirus and increases the spread of the virus. The single strand makes the novel coronavirus more likely to mutate, thereby increasing the difficulty of drug treatment. The main transmission routes of the novel coronavirus include respiratory droplet transmission, contact transmission, aerosol and fecal-oral transmission. Epidemiological investigations show that people of all ages may be infected, and most victims are adults, among whom the elderly, the weak and the sick seem to be more likely to be infected.

Respiratory viral infections are clinically very common, but there are few corresponding therapeutic drugs. Commonly used clinical antiviral drugs mainly include oseltamivir, amantadines, ribavirin, ganciclovir and Chinese patent medicines containing Scutellaria baicalensis or andrographolide, and the main prevention methods for respiratory viral infections are vaccine prevention, personal hygiene, disinfection of the external environment and the like. The current treatment and prevention drugs cannot fully meet the current needs for the treatment of respiratory viral infections, and the speed of research and development of related drugs is far lower than the speed of virus transmission and mutation. Currently commonly used disinfection methods include the use of alcohol, 84 disinfectant, etc., but disinfecting the external environment of the human body such as the surrounding air, hands and feet cannot kill viruses that have already entered the human respiratory system, and the only thing the infected people can do is to wait for the viruses to replicate and cause diseases.

### SUMMARY

In view of the research background above, an objective of the present invention is to provide a method for preventing and treating respiratory virus-related diseases by inactivating respiratory viruses. In order to achieve this objective, the present invention provides the following technical schemes:

According to a first aspect of the present invention, a composition for inhibiting respiratory viruses is provided, where acetic acid is used as an active ingredient in the composition.

The research of the present invention proves that hydrogen ions can inactivate viruses adhering to the human respiratory tract, and acetic acid, as a weak acid, can provide hydrogen ions in a dose of inactivating respiratory viruses at a tolerable concentration, thereby inactivating viruses in the human respiratory tract and preventing and treating respiratory viral infectious diseases.

In addition, the composition includes other auxiliary therapeutic ingredients, such as ethanol, oxygen and anti-allergy ingredients. Ethanol can help inactivate viruses and bacteria and can also help relieve the sensory stimulation of the patient caused by acetic acid. In addition, when acetic acid is used for treatment, medical oxygen is mixed to increase the blood oxygen concentration of the patient to strengthen the cardiopulmonary function of the patient, thereby improving the effect of inactivating respiratory viruses.

According to a second aspect of the present invention, an inhalant for inhibiting respiratory viruses is provided, where the inhalant includes the composition according to the first aspect.

The inhalant of the present invention also includes a composition scheme containing anti-allergy ingredients. Due to individual differences, different people have different tolerances to the ingredients of the above composition. In order to eliminate the possibility of drug allergy and ensure the safety of medication, the inhalant also includes anti-allergy ingredients.

According to a third aspect of the present invention, a respiratory virus prevention and treatment method is provided, where the prevention and treatment method includes inhalation of the inhalant for inhibiting respiratory viruses according to the second aspect.

It is believed through the research of the present invention that in the acetic acid steam at high temperature, acetic acid molecules can ionize to produce more hydrogen ions, and hydrogen ions are the main virus inactivating substances. As the temperature of the steam increases, the ionization output is also increased, and the kinetic energy is also increased substantially. Therefore, increasing the inhalation temperature of the steam as much as possible can further improve the effect of inactivating viruses in the respiratory tract.

According to a fourth aspect of the present invention, a novel coronavirus pneumonia prevention and treatment method is provided, where the method includes the steps of the respiratory virus prevention and treatment method according to the third aspect.

In addition, the novel coronavirus pneumonia treatment method provided by the present invention includes the use of the inhalant in combination with anti-novel coronavirus pneumonia drugs, where α-interferon and the like can be mixed with the inhalant and administrated in an inhalation way; or oral administration or intravenous injection of other anti-novel coronavirus pneumonia drugs is carried out while the inhalant is used for treatment.

According to a fifth aspect of the present invention, application of the composition according to the first aspect in inhibiting fungi and/or bacteria in vitro is provided.

The composition provided by the present invention can also be applied in inhibiting fungi and bacteria in vitro and in the respiratory tract to prevent fungi or bacteria from adhering to the surface of the skin or respiratory tract to cause diseases. The method for preventing respiratory fungi and bacteria is the same as that of the third aspect. The method for inhibiting fungi and bacteria on the skin surface includes the use of the high-temperature steam described in the second aspect to fumigate the skin surface.

The beneficial effects of one or more of the above technical schemes are:

The composition and inhalant provided by the present invention also have a good effect of killing viruses entering the respiratory system, and can be used for prevention and treatment as well as environmental disinfection. It is proved through experiments that those who use this method for disinfection for about 3 minutes once per day will not be infected with the novel coronavirus; and those who have been infected with the novel coronavirus will recover within 4 days by using this method at the early stage.

In addition, the inhalant mentioned in the present invention has extremely low costs. According to the current price level, each person can be protected from infection with only RMB 0.1 yuan per day, and each patient can be cured with less than RMB 10 yuan.

The inhalation disinfection method mentioned in the present invention does not have any toxic or side effects on the human body. After killing the viruses, the high-temperature hydrogen ions in the human body can be reduced to normal body temperature in about 0.1 second, form weakly acidic acetic acid with acetate ions, and be excreted with the sputum.

### DETAILED DESCRIPTION

It should be noted that, the following detailed descriptions are exemplary, and are intended to provide a further description to the present invention. Unless otherwise specified, all technical and scientific terms used herein have the same meaning as commonly understood by those of ordinary skill in the art to which the present invention belongs.

It should be noted that terms used herein are only for the purpose of describing specific implementations and are not intended to limit the exemplary implementations of the present invention. As used herein, the singular form is intended to include the plural form, unless the context clearly indicates otherwise. In addition, it should further be understood that terms “comprise” and/or “include” used in this specification indicate that there are features, steps, operations, devices, components, and/or combinations thereof.

As introduced in the related art, in view of the current situation that anti-respiratory virus drugs cannot satisfy clinical applications, in order to solve the above technical problems, the present invention provides an inhalant for inhibiting respiratory viruses and a treatment method.

According to a first aspect of the present invention, a composition for inhibiting respiratory viruses is provided, where acetic acid is used as an active ingredient in the composition.

The effects of inhibiting respiratory viruses of the present invention include directly inhibiting or killing viruses, reducing virus activity, interfering with virus adherence, preventing viruses from entering cells, inhibiting virus transcription and copying processes or inhibiting virus release.

Preferably, the state in which acetic acid inhibits the activity of respiratory viruses is a steam state, specifically, a high-temperature steam state of an acetic acid solution; that is to say, in the composition, the active ingredient for inhibiting respiratory viruses is high-temperature steam of an acetic acid solution.

It has been proved that the composition of the present invention is administered by inhalation. Based on this administration method, the above composition can directly act on viruses adhered to the respiratory tract and oral mucosa surface, and has a direct inactivating effect, which is better than the effect of inactivating viruses in the interior and interlayer of respiratory mucosal cells.

Further preferably, the steam acting on viruses is at 50° C. or above.

Preferably, the composition also includes one or any mixture of ethanol and oxygen.

Further, the oxygen is medical oxygen.

In some implementations of the present invention, the composition includes acetic acid.

In some implementations of the present invention, the composition includes acetic acid and oxygen.

In some implementations of the present invention, the composition includes acetic acid and ethanol.

In some implementations of the present invention, the composition includes acetic acid, oxygen and ethanol.

Preferably, the composition also includes anti-inflammatory drugs, antiviral drugs and/or immunomodulatory drugs.

According to a second aspect of the present invention, an inhalant for inhibiting respiratory viruses is provided, where the inhalant includes the composition according to the first aspect.

Preferably, the inhalant includes an acetic acid solution, and the concentration of the acetic acid solution is 0.01%-10% (mass fraction).

Preferably, the inhalant includes a mixed solution of acetic acid and ethanol. In the mixed solution, the concentration of acetic acid is 0.01%-10% (mass fraction), and the concentration of ethanol is 0.01%-10% (mass fraction).

Preferably, the inhalant further includes oxygen.

Further preferably, the inhalant includes high-temperature steam of an acetic acid aqueous solution and oxygen.

In some specific implementations of the above preferred technical schemes, the high-temperature steam of the acetic acid aqueous solution and oxygen in the inhalant are not doped with each other and are inhaled in a certain use order.

In some other implementations, the steam of the acetic acid aqueous solution and oxygen are mixed in a certain volume ratio and inhaled for use at the same time.

Further preferably, the inhalant includes high-temperature steam of an acetic acid and ethanol aqueous solution and oxygen.

In some specific implementations of the above preferred technical schemes, the high-temperature steam of the acetic acid and ethanol aqueous solution and oxygen are not doped with each other and are inhaled in a certain use order.

In some other implementations, the high-temperature steam of the acetic acid and ethanol aqueous solution and oxygen are mixed in a certain volume ratio and inhaled for use at the same time.

Preferably, the inhalant further includes anti-allergy ingredients.

Further preferably, the anti-allergy ingredients include antihistamine drugs, allergic reaction mediator release-blocking drugs, histamine desensitizers, leukotriene receptor antagonists, drugs for inhibiting antigen-antibody reactions and drugs for alleviating or controlling allergy symptoms.

Further, the antihistamine drugs include but are not limited to diphenhydramine, promethazine and chlorpheniramine.

Further, the allergic reaction mediator release-blocking drugs include but are not limited to sodium cromoglycate and ketotifen.

Further, the histamine desensitizers include but are not limited to betahistine, small-dose histamine diluent and dust mite injection.

Further, the leukotriene receptor antagonists include but are not limited to montelukast and zafirlukast.

Further, the drugs for inhibiting antigen-antibody reactions are adrenal glucocorticoids, immunosuppressants and the like.

Further, the drugs for alleviating or controlling allergy symptoms include but are not limited to smooth muscle antispasmodics such as salbutamol and drugs for reducing edema caused by allergies such as calcium gluconate.

In the above technical schemes regarding the anti-allergy ingredients, the anti-allergy ingredients are added to the acetic acid and ethanol aqueous solution according to the clinical use concentration, where the leukotriene receptor antagonists such as montelukast and zafirlukast are more suitable for respiratory allergies and are added to the inhalant as more preferred ingredients.

In some specific implementations of the above preferred technical schemes, in the inhalant, the high-temperature steam of an acetic acid, ethanol and anti-allergy agent aqueous solution and oxygen are mixed in a certain volume ratio and inhaled for use at the same time.

According to a third aspect of the present invention, a respiratory virus prevention and treatment method is provided, where the prevention and treatment method includes inhalation of the inhalant for inhibiting respiratory viruses according to the second aspect.

Preferably, the respiratory viruses include but are not limited to rhinoviruses, coronaviruses, enteroviruses, adenoviruses and respiratory syncytial viruses.

Further preferably, the coronaviruses include but are not limited to SARS-CoV and 2019-nCoV.

Preferably, the inhalation temperature of the inhalant is relatively high and can be tolerated by the human body. It is proved by the present invention that the inhalation temperature of the inhalant is 50° C. or above.

Further preferably, a method of using the inhalant is as follows: a part of the inhalant which is in liquid form at room temperature is heated and boiled to produce steam, a patient needs to continuously inhale the steam for a period of time, and the inhalation temperature of the steam should be maintained at 50° C. or above with the high temperature that the human body can withstand as the upper limit.

In some implementations of the above preferred technical schemes, the inhalation time of the steam is 1-20 min/d.

In some implementations of the above preferred technical schemes, the concentration of acetic acid in the inhalant is determined according to the tolerance of a patient. It is recommended to use a 0.01%-2% acetic acid solution for children and elderly patients and an acetic acid solution with the concentration of 0.5%-5% for adult patients to achieve the best therapeutic effects.

In some implementations of the above preferred technical schemes, the inhalant is a mixed aqueous solution of acetic acid and ethanol, where the ratio of acetic acid to ethanol is 1:(0.1-2).

In an implementation with a good effect of the present invention, the inhalant is an acetic acid aqueous solution, the acetic acid aqueous solution is heated to boil, and a patient continuously inhales the steam of the acetic acid aqueous solution at 50° C. or above for 1-10 min for treatment.

In an implementation with a good effect of the present invention, the inhalant is an acetic acid and ethanol aqueous solution. The acetic acid and ethanol aqueous solution is heated to boil, and a patient continuously inhales the steam of the acetic acid and ethanol aqueous solution at 50° C. or above for 5-10 min for treatment, where in the acetic acid and ethanol aqueous solution, the mass ratio of acetic acid to ethanol is 1:1.

In the above series of implementations, inhalation of oxygen is also included for auxiliary therapy. The oxygen is medical oxygen. The patient inhales the steam and oxygen for a period of time respectively, or mixes the steam and oxygen before inhalation.

Preferably, the prevention and treatment method also includes using other drugs at the same time for treatment. The other drugs include but are not limited to neuraminidase inhibitors, steroidal anti-inflammatory drugs, non-steroidal anti-inflammatory drugs, antibiotics, immunostimulants, immunomodulators, nucleoside antiviral agents, nucleotide antiviral agents, anti-fibrosis drugs, caspase inhibitors, creatinine 5′-monophosphate dehydrogenase inhibitors and viral enzyme inhibitors.

According to a fourth aspect of the present invention, a novel coronavirus pneumonia prevention and treatment method is provided, where the method includes the steps of the respiratory virus prevention and treatment method according to the third aspect.

Preferably, the prevention and treatment method is applied to prevention or treatment of novel coronavirus pneumonia.

Preferably, the prevention and treatment method also includes a method of combined use with anti-novel coronavirus pneumonia drugs. The anti-novel coronavirus drugs include but are not limited to α-interferon, lopinavir, ritonavir, ribavirin, favipiravir, chloroquine phosphate or remdesivir.

The novel coronavirus often enters the host body by adhering to the eyes, nose, mouth, hands and other parts of human to cause diseases. By using the methods of the present invention, the eyes, nose, mouth and other parts which are difficult to disinfect can be effectively disinfected. A user only needs to move his or her face close to the steam of the acetic acid solution and inhale the steam for a period of time to achieve a comprehensive disinfection effect on the eyes, nose and mouth. For users who may be exposed to the novel coronavirus, a face mask inhalation method can also be adopted. After mixed with oxygen, the acetic acid steam is introduced into a face mask to disinfect the entire face.

According to a fifth aspect of the present invention, application of the composition according to the first aspect in inhibiting fungi and/or bacteria in vitro is provided.

Preferably, the part in vitro includes the skin surface and the surface of a cavity communicating with the outside; further preferably, the part in vitro includes the skin surface and the respiratory tract.

The fungi include common superficial Bacillus fungi on the skin surface such as Trichophyton, Epidermophyton and Microsporum, deep infection fungi such as Candidas and Cryptococcus neoformans, and other pulmonary system infectious fungi such as Pneumocystis.

The bacteria are preferably respiratory bacteria, which include but are not limited to one or any mixture of Mycobacterium tuberculosis, Corynebacterium diphtheria, Legionella pneumophila, Haemophilus influenzae, Bordetella pertussis, Mycoplasma pneumoniae and Chlamydia pneumoniae.

To make a person skilled in the art understand the technical solutions of the present invention more clearly, the technical solutions of the present invention are described below with reference to specific embodiments.

### Embodiment 1

In this embodiment, an inhalant for inhibiting respiratory viruses was provided. The inhalant was a 2.5% acetic acid solution. A use method of the inhalant was as follows: the inhalant was heated to boil, a patient needed to inhale the steam produced during boiling and fresh air in turn, the inhalation temperature of the steam should be kept at 50° C. or above, and the patient needed to inhale the steam at high temperature as far as the he/she can tolerate.

The treatment frequency for preventing respiratory viruses was once a day, and the inhalation time was 3 min. The treatment frequency for treatment of diseases caused by respiratory viruses was three times a day, and the recommended inhalation time was 5 min each time.

### Embodiment 2

In this embodiment, an inhalant for inhibiting respiratory viruses was provided. The inhalant was a 2.5% mixed aqueous solution of acetic acid and ethanol (the concentrations of acetic acid and ethanol were both 2.5%). A use method of the inhalant was as follows: the inhalant was heated to boil, a patient needed to inhale the steam produced during boiling and fresh air in turn, and the inhalation temperature of the steam should be kept at 50° C. or above.

The treatment frequency for preventing respiratory viruses was once a day, and the inhalation time was 3 min. The treatment frequency for treatment of diseases caused by respiratory viruses was 3-6 times a day, and the recommended inhalation time was 5 min each time.

### Embodiment 3

In this embodiment, an inhalant for inhibiting respiratory viruses was provided. The inhalant included medical oxygen and a 2.5% mixed aqueous solution of acetic acid and ethanol (the concentrations of acetic acid and ethanol were both 2.5%). A use method of the inhalant was as follows: the inhalant was heated to boil, a patient needed to inhale a mixture of the steam produced during boiling and the medical oxygen for a period of time, and the inhalation temperature of the steam should be kept at 50° C. or above.

The treatment frequency for preventing respiratory viruses was once a day, and the vapor inhalation time was 3 min. The treatment frequency for treatment of diseases caused by respiratory viruses was three times a day, and the recommended steam inhalation time was 5 min each time.

### Embodiment 4

In this embodiment, an inhalant for prevention and treatment of novel coronary pneumonia was provided. The inhalant included medical oxygen and a 2.5% mixed aqueous solution of acetic acid and ethanol (the concentrations of acetic acid and ethanol were both 2.5%). A use method of the inhalant was as follows: the inhalant was heated to boil, a patient needed to inhale the steam produced during boiling and the medical oxygen in turn, and the inhalation temperature of the steam should be kept at 50° C. or above.

The treatment frequency for preventing novel coronary pneumonia was once a day, and the steam inhalation time was 3 min. The treatment frequency for treatment of novel coronary pneumonia was three times a day, and the recommended steam inhalation time was 5 min each time.

### Embodiment 5

In this embodiment, a novel coronary pneumonia treatment method was provided. Different from the method in Embodiment 4, in this embodiment, atomized α-interferon was also inhaled at the same time.

### Embodiment 6

In this embodiment, an inhalant for prevention and treatment of novel coronary pneumonia was provided. The inhalant included medical oxygen and a 2.5% mixed aqueous solution of acetic acid, ethanol and montelukast (the concentrations of acetic acid and ethanol were both 2.5%). A use method of the inhalant was as follows: the inhalant was heated to boil, a patient needed to inhale the steam produced during boiling and the medical oxygen in turn, and the inhalation temperature of the steam should be kept at 50° C. or above.

The treatment frequency for preventing novel coronary pneumonia was once a day, and the steam inhalation time was 3 min. The treatment frequency for treatment of novel coronary pneumonia was three times a day, and the recommended steam inhalation time was 5 min each time.

### Embodiment 7

In this embodiment, an inhalant for prevention and treatment of respiratory bacterial and fungal infections was provided. The inhalant included medical oxygen and a 2.5% mixed aqueous solution of acetic acid, ethanol and montelukast (the concentrations of acetic acid and ethanol were both 2.5%). A use method of the inhalant was as follows: the inhalant was heated to boil, a patient needed to inhale the steam produced during boiling and the medical oxygen in turn, and the inhalation temperature of the steam should be kept at 50° C. or above.

The treatment frequency for preventing bacterial and fungal infections was once a day, and the steam inhalation time was 3 min. The treatment frequency for treatment of bacterial and fungal infections was three times a day, and the recommended steam inhalation time was 5 min each time.

### Embodiment 8

40 patients infected with novel coronary pneumonia were collected and randomly divided into two groups. The control group was treated with a conventional novel coronary pneumonia treatment method, and the experimental group was treated with the method according to Embodiment 1. After treatment for a period of time, the cure rates of the patients in the control group and the experimental group were tested respectively according to the provisions in the Diagnosis and Treatment Plan for Pneumonia Infected by Novel Coronavirus (Trial Fifth Edition): the body temperature returns to normal for 3 days or more, respiratory symptoms are improved significantly, lung imaging shows obvious absorption of inflammation, and two consecutive nucleic acid tests for respiratory pathogens are negative (the sampling interval is at least 1 day).

### Embodiment 9

From 2019 to 2020, 20 influenza patients were recruited as volunteers and the treatment method described in Embodiment 1 was used for treatment. A treatment course was 3 days, the cure rate was 100%, and the cure time was one treatment course, where the temperature of patients with high fever can drop to normal level on the second day of treatment.

The foregoing descriptions are merely preferred embodiments of the present invention but are not intended to limit the present invention. The present invention may include various modifications and changes for a person skilled in the art. Any modification, equivalent replacement, or improvement made within the spirit and principle of the present invention shall fall within the protection scope of the present invention.

1. A composition for inhibiting respiratory viruses, wherein acetic acid is used as an active ingredient in the composition; the active ingredient for inhibiting respiratory viruses in the composition is high-temperature steam of an acetic acid solution; and the steam acting on viruses is at 50° C. or above

2. The composition for inhibiting respiratory viruses according to claim 1, wherein the composition further comprises one or any mixture of ethanol and oxygen; further, the composition comprises acetic acid;
or, the composition comprises acetic acid and oxygen;
or, the composition comprises acetic acid and ethanol;
or, the composition comprises acetic acid, oxygen and ethanol;
or, the composition also comprises anti-inflammatory drugs, antiviral drugs and immunomodulatory drugs.

3. An inhalant for inhibiting respiratory viruses, wherein the inhalant comprises the composition according to claim 1.

4. The inhalant for inhibiting respiratory viruses according to claim 3, wherein the inhalant comprises an acetic acid solution, and the concentration of the acetic acid solution is 0.01%-10%;
or, the inhalant is a mixed solution of acetic acid and ethanol, and in the mixed solution, the concentration of acetic acid is 0.01%-10%, and the concentration of ethanol is 0.01%-10%.

5. The inhalant for inhibiting respiratory viruses according to claim 4, wherein the inhalant comprises an acetic acid solution and oxygen;
further, the steam of the acetic acid aqueous solution and oxygen in the inhalant are not doped with each other and are inhaled in a certain use order; and
further, the steam of the acetic acid aqueous solution and oxygen are mixed in a certain volume ratio and inhaled for use at the same time.

6. The inhalant for inhibiting respiratory viruses according to claim 4, wherein the inhalant also comprises anti-allergy ingredients that comprise antihistamine drugs, allergic reaction mediator release-blocking drugs, histamine desensitizers, leukotriene receptor antagonists, drugs for inhibiting antigen-antibody reactions and drugs for alleviating or controlling allergy symptoms;
the antihistamine drugs comprise diphenhydramine, promethazine and chlorpheniramine;
the allergic reaction mediator release-blocking drugs comprise sodium cromoglycate and ketotifen;
the histamine desensitizers comprise betahistine, small-dose histamine diluent and dust mite injection;
the leukotriene receptor antagonists comprise montelukast and zafirlukast;
the drugs for inhibiting antigen-antibody reactions are adrenal glucocorticoids and immunosuppressants; and
the drugs for alleviating or controlling allergy symptoms comprise smooth muscle antispasmodics and drugs for reducing edema caused by allergies.

7. A respiratory virus prevention and treatment method, wherein the prevention and treatment method comprises inhalation of the inhalant for inhibiting respiratory viruses according to claim 3;
the respiratory viruses comprise rhinoviruses, coronaviruses, enteroviruses, adenoviruses and respiratory syncytial viruses; further, the coronaviruses comprise SARS-CoV and 2019-nCoV;
the inhalation temperature of the inhalant is 50° C. or above; further, a use method of the inhalant is as follows: a part of the inhalant which is in liquid form at room temperature is heated and boiled to produce steam, a patient needs to continuously inhale the steam for a period of time, and the temperature of the steam should be maintained at 50° C. or above with the high temperature that the human body can withstand as the upper limit; further, the steam inhalation time is 1-20 min/d;
further, the concentration of acetic acid in the inhalant is determined according to the tolerance of a patient, a 0.01%-2% acetic acid solution is used for children and elderly patients for treatment, and an acetic acid solution with the concentration of 0.5%-5% is used for adult patients for treatment; and
further, the inhalant is a mixed aqueous solution of acetic acid and ethanol with a ratio of acetic acid to ethanol being 1:(0.1-2).

8. The respiratory virus prevention and treatment method according to claim 7, wherein the prevention and treatment method also comprises using other drugs at the same time for treatment, and the other drugs comprise neuraminidase inhibitors, steroidal anti-inflammatory drugs, non-steroidal anti-inflammatory drugs, antibiotics, immuno stimulants, immunomodulators, nucleoside antiviral agents, nucleotide antiviral agents, anti-fibrosis drugs, caspase inhibitors, creatinine 5′-monophosphate dehydrogenase inhibitors and viral enzyme inhibitors.

9. A novel coronavirus pneumonia prevention and treatment method, wherein the method comprises the steps of the respiratory virus prevention and treatment method according to claim 7;
the prevention and treatment method is applied to prevention or treatment of novel coronavirus pneumonia; and
the prevention and treatment method also comprises a method of combined use with anti-novel coronavirus pneumonia drugs, and the anti-novel coronavirus drugs comprise α-interferon, lopinavir, ritonavir, ribavirin, favipiravir, chloroquine phosphate or remdesivir.

10. A method comprising applying the composition according to claim 1 in inhibiting fungi and/or bacteria in vitro, wherein
the part in vitro comprises the skin surface and the surface of a cavity communicating with the outside;
the fungi comprise Trichophyton, Epidermophyton, Microsporum, Candidas, Cryptococcus neoformans and Pneumocystis; and
the bacteria are respiratory bacteria, which comprise one or any mixture of Mycobacterium tuberculosis, Corynebacterium diphtheria, Legionella pneumophila, Haemophilus influenzae, Bordetella pertussis, Mycoplasma pneumoniae and Chlamydia pneumoniae.
